(19) United States
(12) Patent Application Publication
(10) Pub. No.: US 2010/0030250 A1
(43) Pub. Date: Feb. 4, 2010

(54) CATHETER, IN PARTICULAR PTCA CATHETER

(75) Inventor: Boris Warnack, Mountain View, CA (US)

Correspondence Address: LUCE, FORWARD, HAMILTON & SCRIPPS, 11988 EL CAMINO REAL, SUITE 200, SAN DIEGO, CA, 92130, US

(73) Assignee: ABBOTT LABORATORIES VASCULAR ENTERPRISES LIMITED, Dublin (IE)

(21) Appl. No.: 12/063,662

(22) PCT Filed: Aug. 17, 2006
(86) PCT No.: PCT/EP06/08117

§ 371 Date: May 11, 2009

Related U.S. Application Data
(60) Provisional application No. 60/709,627, filed on Aug. 19, 2005.

(51) Int. Cl. A61M 25/10

(52) U.S. Cl. 606/194

(57) ABSTRACT

Catheter 1, in particular PTCA catheter, comprising an outer tube 2; an inner guide wire tube 3 within the outer tube 2, said inner guide wire tube 3 having a tip 4; a first and a second marker 5, 6 fixed to the guide wire tube 3 at a fixed distance b to one another; and a balloon 7 having two sleeves 8, 9 fixed to the outer tube 2 and the inner tube 3, respectively, wherein that the distance b between the markers 5, 6 is set to be at least approximately equal to the distance a between the tip 4 and the marker 5 neighbouring the tip 4.

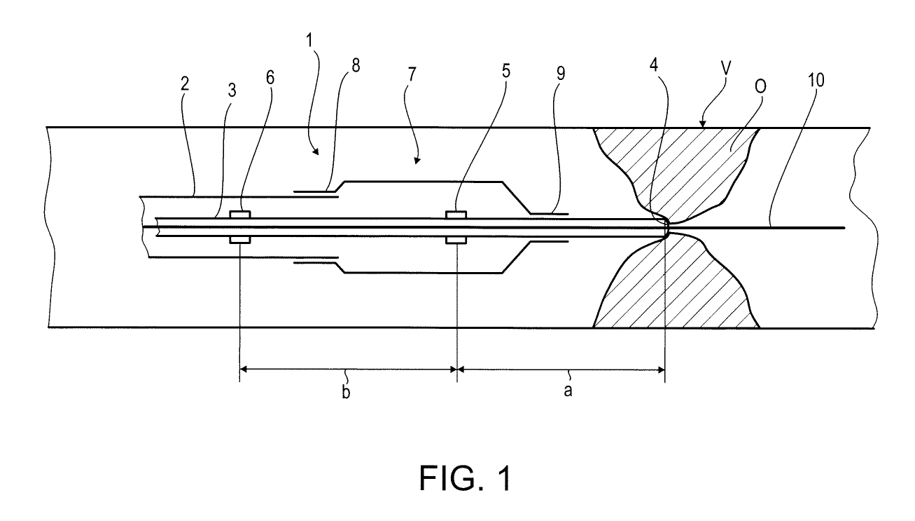

The invention concerns a catheter, in particular a PTCA catheter, according to the preamble part of claim 1.

A catheter according to the preamble part of claim 1 usually includes one or two markers, in particular in form of marker bands, made from radiopaque material to identify the position of the balloon within the anatomy when using X-ray imaging methods. Other parts of the catheter, especially the catheter tip, remain nearly invisible for X-ray imaging.

Catheter markers are well known in the art to enable the physician to track the position of the catheter in the patient's body under X-Ray. As described in U.S. Pat. No. 5,779,731 and U.S. Pat. No. 5,846,199 radiopaque markers can be used to indicate the working length of the balloon of a balloon catheter. U.S. Pat. No. 5,209,730 describes a method for accurate placement of a balloon catheter across a stenosis employing cooperating radiopaque markers on the guidewire and the catheter. Further, as depicted in U.S. Pat. No. 5,669,932, markers are employed to indicate the position of the stent in a stent delivery catheter. Markers can be made of diverse materials such as radiopaque metal alloys or polymers doped with radiopaque material as described in U.S. Pat. No. 6,540,721. In special cases, however, it is important for the physician not only to know the accurate position of the balloon but also to know whether the tip of the catheter has already entered a certain region of the lumen the catheter has been introduced in, and in particular whether the catheter has already entered a tight lesion, or not.

It is therefore an object of the present invention to provide a catheter according to the preamble part of claim 1 that is able to facilitate the handling thereof, especially in terms of the identification of the positioning of the catheter tip.

The solution of this object is achieved by the features of claim 1.

The catheter according to the present invention makes it possible to give a tool to the physician to judge whether the tip of the catheter has already entered a certain region of a vessel, especially an occlusion, or not. For the treatment of chronic total occlusion (CTO), the tip and the complete distal part of the catheter has to be very thin and low in diameter, to offer the highest possibility to pass the lesion. As it is not possible to make the tip of a catheter from a radiopaque material or to fill the polymer with a radiopaque agent as this would inevitably thicken the catheter, what would not be acceptable for the reasons given hereinbefore, the catheter of the present application is especially advantageous as it can be kept very thin.

This specific advantage is achieved by the invention and provides that there is a second radiopaque marker, especially proximal of the balloon, that is located proximally at the same distance from the balloon marker neighbouring the tip in the distal direction. This second proximal marker serves as a measuring marker. The physician can thus judge the distance from the first distal marker to the tip by using the known distance between the measuring marker and the first marker that is advantageously located in the balloon.

The markers can be of various materials, such as solid or wired platinum, iridium, gold tantalum etc. The proximal measuring marker should have a different geometry than the first marker (balloon marker) to avoid mixing up and to avoid a balloon dilatation at a wrong position.

The dependent claims contain advantageous embodiments of the present application.

Furthermore, the invention also concerns the use of the catheter according to the present invention in treating vessels, in particular blood vessels, that are blocked especially by a total occlusion. In general, the catheter according to the present invention can be used as a usual PTCA catheter but furnishes the advantage that the physician using the inventive catheter can judge where the catheter tip is located within the vessel by using the known distance between the measuring marker and the balloon marker in order to evaluate where the more or less invisible catheter tip is positioned.

Further features and advantages of the present application will become apparent from the description of the single FIGURE of the drawings.

This single FIGURE is a schematically simplified illustration of a catheter 1, especially in the form of a PTCA catheter. A catheter 1 comprises an outer tube 2 and an inner guide wire tube 3 that is located within the outer tube 2, in particular concentrically oriented with respect to said outer tube 2.

The inner guide wire tube 3 guiding the guide wire 10 includes a tip 4. The single figure of the drawing shows the catheter 1 being located in a vessel V with a tip 4 abutting against a total occlusion O of said vessel V.

For the purpose of visualization of the positioning of the catheter 1 within said vessel V, e.g. by X-ray, the catheter 1 comprises two radiopaque markers 5 and 6 fixed to the inner guide wire tube 3. Marker 5 serves as balloon marker and is centrically positioned in the balloon. The tip 4, however, is virtually invisible. So, according to the principles of the present application, the two markers 5 and 6 are disposed with respect to one another at a known distance b that is set to be equal to a distance a between marker 5 and tip 4.

According to the depicted embodiment, the marker 5 is a first marker being disposed within the balloon 7 that, of course, is not yet inflated in the condition shown in the figure. The marker 6 is disposed proximal of the balloon 7 at said known distance b.

In order to be able to guide the catheter 1 through the occlusion O, it is not possible to dispose another marker at or nearby tip 4 as this would unacceptably thicken the catheter 1.

As, however, the distances a and b are equal or at least nearly equal, the physician can evaluate the positioning of the tip 4 upon visualization of the markers 5 and 6 as he knows that these markers 5 and 6 are disposed with respect to one another at the known distance b.

### REFERENCE NUMERALS

1 Catheter

2 Outer tube

3 Inner guide wire tube

4 Tip

5 First marker (balloon marker)

6 Second marker (proximal marker)

7 Balloon

8 Sleeve (fixed to outer tube 2)

9 Sleeve (fixed to inner tube 3)

10 Guide Wire

b known distance between markers 5 and 6

a distance between marker 5 and tip 4

V Vessel

O Occlusion

(The catheter described as a PTCA catheter comprising a balloon can also be a catheter without a balloon according to the principles of the present invention)

1) Catheter having a distal portion bearing a functional unit and a catheter tip distal of the functional unit,
the distal portion of the catheter comprising a distal marker and a proximal marker,
wherein the distance between the distal end of the catheter tip and the center of the distal marker is approximately equal to the distance between the center of the distal marker and the center of the proximal marker.

2) Catheter according to claim 1 wherein the functional unit is a balloon.

3) Catheter according to claim 1 wherein the distal marker is located in the center of the functional unit.

4) Catheter according to claim 1 wherein the distal and the proximal marker are visually distinguishable from each other under x-Ray.

5) Use of a catheter according to claim 2 for treatment of coronary total occlusions.

6) Method of treating a coronary total occlusion comprising the steps of
a. providing a catheter having a distal portion bearing a balloon and a catheter tip distal of the balloon, the distal portion of the catheter comprising a distal marker and a proximal marker, the distal marker being located in the center of the balloon; wherein the distance between the distal end of the catheter tip and the center of the distal marker is approximately equal to the distance between the center of the distal marker and the center of the proximal marker;
b. inserting the catheter into a patient's blood vessels;
c. advancing at least a distal portion of the catheter across the occlusion in the vessel;
d. positioning the balloon within the occlusion;
e. inflating the balloon;
f. deflating the balloon; and
g. retracting the catheter from the vessel.
